(19) United States
(12) Patent Application Publication
(10) Pub. No.: US 2024/0197249 A1
(43) Pub. Date: Jun. 20, 2024

(54) TECHNIQUES FOR COLLECTING BIOIMPEDANCE DATA USING A WEARABLE DEVICE

(71) Applicant: Oura Health Oy, Oulu (FI)

(72) Inventors: Heli Tuulia Koskimäki, Oulu (FI); Pauli Ohukainen, Oulu (FI); Jussi Petteri Järvelä, Kempele (FI); Juha-Pekka Syrjälä, Oulu (FI); Veli-Pekka Kullervo Halme, Oulu (FI)

(21) Appl. No.: 18/516,094

(22) Filed: Nov. 21, 2023

Related U.S. Application Data
(60) Provisional application No. 63/387,846, filed on Dec. 16, 2022.

(51) Int. Cl. A61B 5/00; A61B 5/053

(52) U.S. Cl. CPC A61B 5/6826; A61B 5/053; A61B 5/4806; A61B 5/4857; A61B 5/7285; A61B 5/7435; A61B 5/7475; A61B 2562/0209; A61B 2562/043

(57) ABSTRACT

Methods, systems, and devices for bioimpedance measurements using a wearable device are described. The method may include generating a first electrical signal using a first electrode of a wearable ring device, and receiving the first electrical signal using a second electrode of the wearable ring device. The first electrode or the second electrode may be disposed within an inner circumferential surface of the wearable ring device. Further, the method may include determining first bioimpedance data associated with a user based on a comparison of the first electrical signal generated by the first electrode and the first electrical signal received by the second electrode, and causing a graphical user interface (GUI) of a user device to display a message associated with the first bioimpedance data.

### CROSS REFERENCE

The present application for patent claims the benefit of U.S. Provisional Application No. 63/387,846 by Koskimäki et al., entitled “TECHNIQUES FOR COLLECTING BIOIMPEDANCE DATA USING A WEARABLE DEVICE,” filed Dec. 16, 2022, which is assigned to the assignee hereof and expressly incorporated by reference herein.

### FIELD OF TECHNOLOGY

The following relates to wearable devices and data processing, including techniques for collecting bioimpedance data using a wearable device.

### BACKGROUND

Some wearable devices may be configured to collect data from a user. For example, some wearable devices may be configured to collect biological data associated with the user. In some examples, biological data of the user may be determined using measured metrics such as bioimpedance. However, some wearable devices may not have a capability to measure bioimpedance.

### BRIEF DESCRIPTION OF THE DRAWINGS

FIG. 1 illustrates an example of a system that supports techniques for collecting bioimpedance data using a wearable device in accordance with aspects of the present disclosure.

FIG. 2 illustrates an example of a system that supports techniques for collecting bioimpedance data using a wearable device in accordance with aspects of the present disclosure.

FIG. 3 illustrate an example of a system that supports techniques for collecting bioimpedance data using a wearable device in accordance with aspects of the present disclosure.

FIG. 4 illustrates an example of a graphical user interface (GUI) that supports techniques for collecting bioimpedance data using a wearable device in accordance with aspects of the present disclosure.

FIGS. 5A and 5B illustrate examples of electrode configurations that support techniques for collecting bioimpedance data using a wearable device in accordance with aspects of the present disclosure.

FIG. 6 illustrates an example of a bioimpedance-time graph that supports techniques for collecting bioimpedance data using a wearable device in accordance with aspects of the present disclosure.

FIG. 7 illustrates a block diagram of an apparatus that supports techniques for collecting bioimpedance data using a wearable device in accordance with aspects of the present disclosure.

FIG. 8 illustrates a block diagram of a wearable device manager that supports techniques for collecting bioimpedance data using a wearable device in accordance with aspects of the present disclosure.

FIG. 9 illustrates a diagram of a system including a device that supports techniques for collecting bioimpedance data using a wearable device in accordance with aspects of the present disclosure.

FIGS. 10 through 12 illustrate flowcharts showing methods that support techniques for collecting bioimpedance data using a wearable device in accordance with aspects of the present disclosure.

### DETAILED DESCRIPTION

In some examples, a wearable device may determine one or more physiological measurements of a user of the wearable device. For example, the wearable device may measure temperature, heart rate, blood pressure, etc. However, such physiological measurements may not provide the user with a complete picture of their overall health. For example, conventional wearable devices configured to acquire temperature and heart rate data may be unable to determine biological measurements related to the user's lifestyle (e.g., body composition of the user or blood content of the user, eating habits, etc.), which is an important aspect of the user's overall health. Determining such biological measurements may allow the wearable device to provide the user with information that may allow the user to improve his or her overall health.

As described herein, a wearable device may utilize bioimpedance measurements to determine biological data for the user of the wearable device. In some examples, the wearable device may be an example of a ring and may be worn around a finger of the user. Further, the wearable device may include at least a first electrode and a second electrode. Using the first electrode, the wearable device may generate an electrical signal, and may receive the electrical signal using the second electrode. In some examples, the electrical signal may travel through the finger of the user. Upon receiving the electrical signal, the wearable device may compare the generated electrical signal and the received electrical signal and determine bioimpedance data associated with the user using the comparison.

If the wearable device detects a change in bioimpedance data (e.g., relative to the user's baseline bioimpedance data, or relative to bioimpedance data collected over some previous time period), the wearable device may determine a change in one or more biological metrics of the user. For example, if the change in the bioimpedance data exceeds a threshold (e.g., baseline threshold, user-specific threshold), the wearable device may determine that the user has consumed a meal high in fat. Further, upon detecting the change in bioimpedance data, the wearable device may provide the user with one or more messages. For example, upon detecting that the user consumed a meal high in fat, the wearable device may generate a message indicating for the user to decrease the amount of fat in the user's diet or to add more fiber to the user's diet. Such techniques may enable a user to make informed decisions on their health, and may provide the user with a more comprehensive picture of their overall health as compared to some conventional wearable devices.

Aspects of the disclosure are initially described in the context of systems supporting physiological and body composition data collection from users via wearable devices. Additional aspects of the disclosure are described in a context of a graphical user interface (GUI), electrode configurations, and a bioimpedance-time graph. Aspects of the disclosure are further illustrated by and described with reference to apparatus diagrams, system diagrams, and flowcharts that relate to techniques for collecting bioimpedance data using a wearable device.

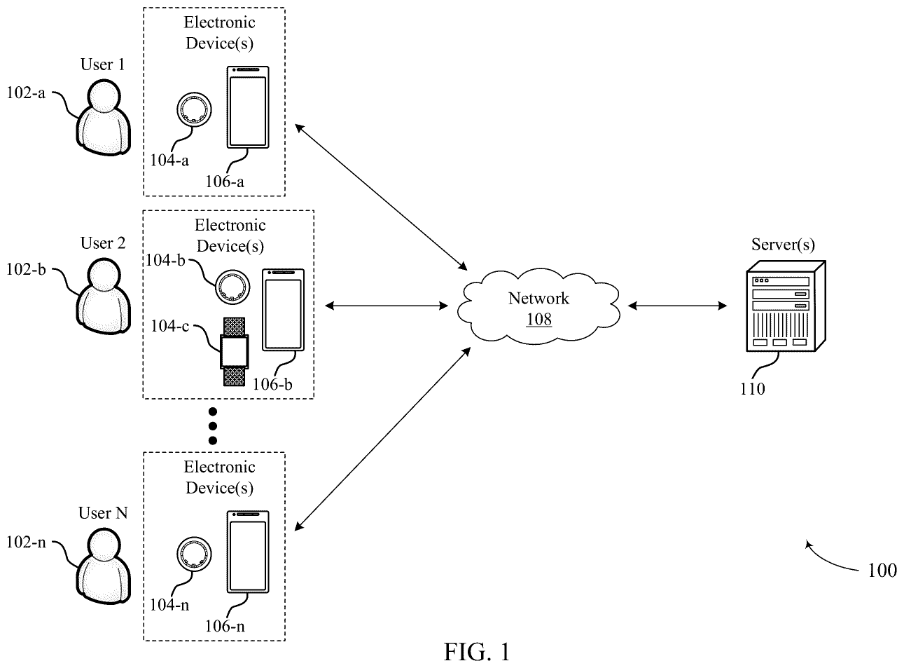

FIG. 1 illustrates an example of a system 100 that supports techniques for collecting bioimpedance data using a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IOT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b or a wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state: 2) circadian rhythms: 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used): 5) multi-day ovarian rhythms in women and spermatogenesis rhythms in men: 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for utilizing bioimpedance data to determine physiological and body composition parameters of the user 102, such as blood glucose level, an amount of body fat, an amount of muscle, HRV, body fluid components, hydration/dehydration, etc. In some examples, the wearable device 104 may include one or more electrodes. For example, the wearable device 104 may include a first electrode and a second electrode. The one or more electrodes may be disposed within an inner circumferential surface of the wearable device 104 creating a signal path through a finger of the user 102. Using the first electrode, the wearable device 104 may generate an electrical signal associated with a first frequency, and the wearable device 104 may receive the electrical signal using the second electrode. The wearable device 104 may compare the electrical signal generated by the first electrode with the electrical signal received by the second electrode and determine bioimpedance data based on the comparison. In particular, the wearable device 104 may compare the generated electrical signal with the received electrical signal to determine the opposition (e.g., impedance) of the flow of the electrical current between the respective electrodes, which may be referred to as “bioimpedance data.”

In some examples, the wearable device 104 may perform multiple bioimpedance measurements. In such examples, if the wearable device 104 detects a change from one measured bioimpedance measurement to the next, the wearable device 104 may also determine a change in one or more biological parameters of the user 102. For example, if the wearable device 104 determines a change in the measured bioimpedance, the wearable device 104 may also determine a change in fat circulated in the blood, a change in body composition (e.g., body fat, muscle mass), or a change in heart rate. In some examples, the wearable device 104 may communicate bioimpedance data to one or both of the server 110 or the user device 106 for further processing or storage. Further, the wearable device 104 may cause a GUI (e.g., of a user device 106) to display a message associated with the bioimpedance data. As an example, if the change in bioimpedance exceeds a threshold, the wearable device 104 may cause the GUI to display a message informing the user 102 that a high fat meal has been consumed.

For the purposes of the present disclosure, the term “physiological,” “physiology,” and like terms, may be used to refer to the function of cells, tissues, organs, and organ systems within the body. Comparatively, the term “body composition” may refer to the relative/absolute amounts or compositions of specific cells or tissues, such as relative/absolute amounts of adipose tissue (e.g., fat), muscle, bone, water, etc. Moreover, the term “biological data” may be used generally to refer to data that refers to the overall biology of the user, and may therefore encompass both physiological and body composition data.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

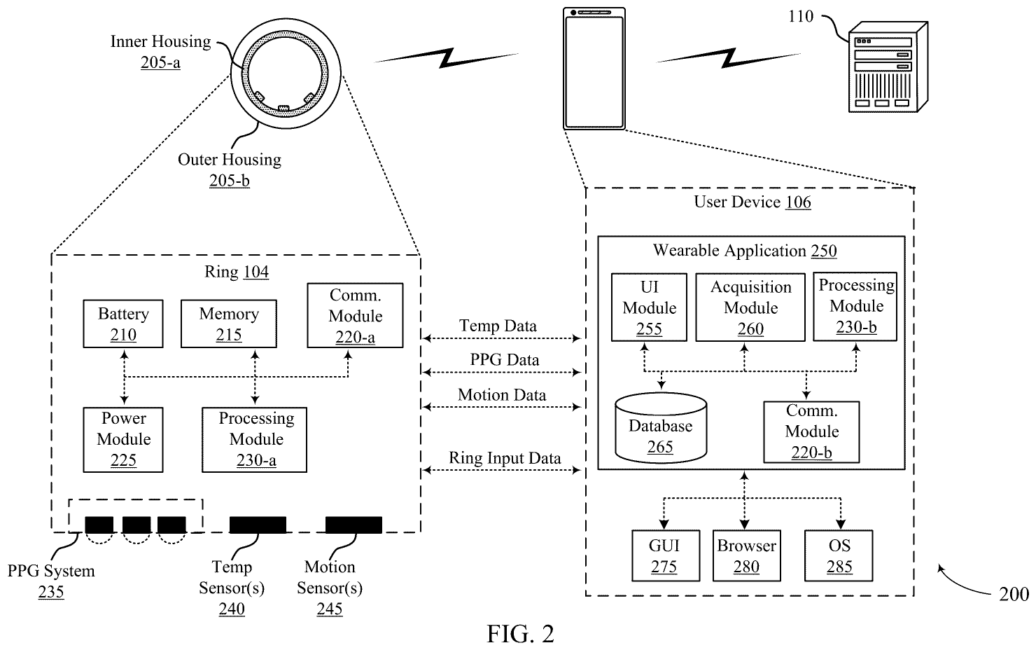

FIG. 2 illustrates an example of a system 200 that supports techniques for collecting bioimpedance data using a wearable device in accordance with aspects of the present disclosure. The system 200 may implement, or be implemented by, a system 100. In particular, the system 200 may illustrate an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a. 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency.” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for measuring bioimpedance data of the user and utilizing the bioimpedance data of the user to determine physiological and body composition parameters of the user (e.g., a blood content parameter, a blood pressure parameter, a glucose parameter, etc.). The ring 104 may include at least two electrodes (e.g., copper electrodes with conductive silver paint). In other cases where the housing 205 of the ring 104 is made of metal, or includes metal components, the metal material of the housing 205 itself may be used as electrodes. An electrode may be described as an electrical conductor. When placed on either side of a conductive material, a pair of electrodes (e.g., a cathode and an anode) may create an electrical current through the conductive material. As an example, a first electrode may be located at or within the inner housing 205-a of the ring 104 and the second electrode may be located at or within the inner housing 205-a of the ring 104 opposite from the first electrode. In such examples, the conductive material between the first electrode and the second electrode may be a finger of the user.

The first electrode and the second electrode may be coupled to one or more components of the ring 104. For example, the first electrode and the second electrode may be coupled to one or more of a voltage source (e.g., a battery 210), a current generator, a voltage meter, a controller, the memory 215, or the processing module 230. Using the one or more components (e.g., the controller), the ring 104 may generate an electrical signal via the first electrode and receive the electrical signal via the second electrode. Further, using the one or more components (e.g., processing module 230), the ring 104 may compare the generated electrical signal and the received electrical signal and determine bioimpedance data (e.g., determine resistance to the flow of the electrical signal). In some examples, to improve the accuracy of bioimpedance data, the ring 104 may incorporate or consider physiological measurements (e.g., PPG data or PPG signals). Upon determining the bioimpedance data, the ring 104 may store the bioimpedance data in the memory 215. Further, the ring 104 may transfer the bioimpedance data to the user device 106 and the user device 106 may display a message associated with the bioimpedance data using the GUI 275.

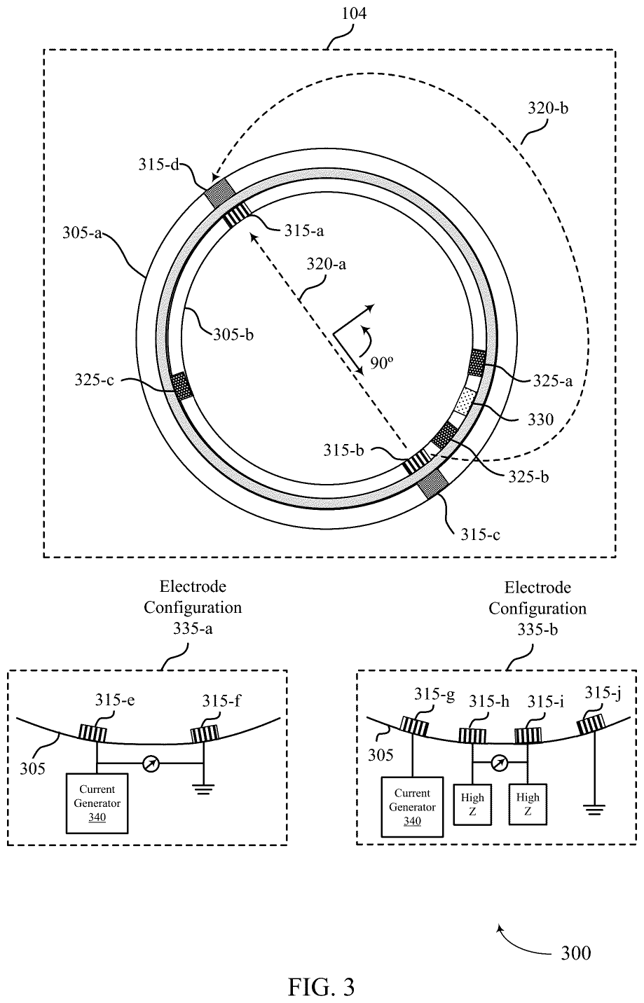

FIG. 3 illustrates an example of a system 300 that supports techniques for collecting bioimpedance data using a wearable device in accordance with aspects of the present disclosure. In some examples, aspects of the system 300 may implement, or be implemented by, aspects of the system 100, and the system 200, or both. For example, the system 300 may include a wearable device 104 which may be an example of a wearable device 104 and a ring 104 as described with reference to FIGS. 1 and 2. In some examples, the wearable device 104 may include multiple components. For example, the wearable device 104 may include an optical transmitter 330 (e.g., LEDs, lasers, or other light-emitting components) and optical receivers 325 (e.g., photodetectors) which may be examples of the one or more sensors included in a PPG system 235 as described with reference to FIG. 2.

As described with reference to FIG. 2, a wearable device 104 may perform one or more physiological measurements. As one example, the wearable device 104 may utilize one or more of the optical transmitters 330 along with at least one optical receiver 325 to measure an oxygen saturation level or an HRV of a user of the wearable device 104. Further, the wearable device 104 may include one or more temperature sensors (not shown) and may utilize the one or more temperature sensors to measure a temperature of the user of the wearable device 104. These physiological measurements may inform the user of different characteristics related to the user's health, and may potentially allow the user to make changes that may improve their overall health.

Other biological measurements that may allow the user to make informed decisions about their health may include body composition measurements and/or measurements for fat content in the blood of the user. Body composition measurements may be indicative of a body fat percentage and/or muscle mass of the user, and fat content in the blood may be indicative of an amount of fat present in the user's blood at one time (which may be based on the user's diet).

In some examples, the body composition of the user or fat content may be measured using bioimpedance. Bioimpedance may indicate how well electrical current runs through different tissues of the body. Different tissues may be associated with different resistivities. For example, fat may have a relatively high resistivity whereas blood may have a lower resistivity. As such, a person with more fat (e.g., in the blood or on the body) will have a higher bioimpedance measurement, whereas a person with less fat may have a lower bioimpedance measurement.

As described herein, the wearable device 104 may measure bioimpedance of the user in order to determine body composition measurements of the user of the wearable device 104. In some examples, the wearable device 104 may include electrodes 315. In one example, the one or more electrodes 315 may be located at or within an inner-housing 305-b (e.g., inner circumferential surface) of the wearable device 104. For example, the wearable device 104 may include an electrode 315-a and an electrode 315-b. Additionally, at least one surface of the one or more electrodes 315 located at the inner-housing 305-b may be exposed (e.g., one surface of the electrodes 315 may be touching the user's finger). Additionally or alternatively, one or more electrodes 315 may be located within an outer-housing 305-a (e.g., outer circumferential surface) of the wearable device 104. Further, at least one surface of the one or more electrodes 315 located at the outer-housing 305-a may be exposed. In some examples, the electrodes 315 may include a conductive material such as copper, graphite, titanium, brass, silver, platinum, etc. Specifically, the electrodes 315 may include copper and the at least one exposed surface of the electrodes 315 may be painted with conductive silver paint.

Further, one or more different combinations of electrodes 315 may be present in the wearable device 104. For example, a first combination of electrodes 315 may include the electrode 315-a and the electrode 315-b. In such a combination, the electrode 315-a may be located opposite from the electrode 315-b along the inner-housing 305-b. That is, the electrode 315-a may be located at least 90 degrees away from the electrode 315-b on the inner-housing 305-b with respect to the x-axis in a counterclockwise or clockwise direction. In the example of FIG. 3, the electrode 315-a is directly opposite or 180 degrees away from the electrode 315-b.

Further, a second combination of electrodes 315 may include one of either the electrode 315-a or the electrode 315-b and one of either the electrode 315-c and the electrode 315-d. Additionally, a third combination of electrodes may include the electrode 315-d and the electrode 315-c. In some examples, the wearable device 104 may activate different combinations of electrodes 315 depending on the biological measurement being made. For example, when measuring a fat content of the blood, the wearable device 104 may activate the first combination of electrodes 315 (e.g., electrodes 315-a and 315-b). Alternatively, when measuring a body composition of the user, the wearable device 104 may activate the second combination of electrodes 315 (e.g., electrodes 315-a and 315-c, or electrodes 315-b and 315-d) or the third combination of electrodes 315 (e.g., electrodes 315-c and 315-d).

In some examples, different combinations of electrodes 315 may generate electrical current through the user's body along different signal paths 320. In order to generate the electrical current, a combination of electrodes 315 may be coupled to one or more voltage sources (e.g., a battery or a capacitor). As one example, the electrode 315-b (e.g., an anode) may be coupled to a positive terminal of the voltage source and the electrode 315-a (e.g., a cathode) may be coupled to a negative terminal of the voltage source or ground. Coupling the electrode 315-b and the electrode 315-a to the one or more voltages sources in such a way may create a signal path 320-a through the user's finger. That is, electrical current may travel through the user's finger from the electrode 315-b to the electrode 315-a along the signal path 320-a.

In another example, the user may interact with an electrode 315 located at or within the outer-housing 305-a to create a signal path 320-b. For example, the user may touch a finger of a hand different from the hand wearing the wearable device 104 to one of the electrodes 315-c or 315-d, thus creating a signal path 320 (e.g., signal path 320-b) between one of the electrode 315-a or the electrode 315-b and one of the electrodes 315-c and the electrode 315-d. Such a signal path 320-b may travel across a chest of the user from one hand of the user to another hand of the user (e.g., electrical current generated by electrode 315-b travels up through the user's hand wearing the wearable device 104, across their chest, down the other arm and through a finger touching the outer electrode 315-d). In another example, the user may interact with more than one electrode 315 located at or within the outer-housing 305-a to create a signal path 320-b. For example, the user may touch a finger to the electrode 315-c and a different finger of the same hand to the electrode 315-d. In such examples, the signal path 320 may travel up one finger of a hand of the user and down another finger of the same hand of the user.

The electrode configuration 335-a and the electrode configuration 335-b may illustrate electrical circuits or electrical components that may support bioimpedance measurements performed by the wearable device 104. In this regard, the electrodes 315 illustrated in the respective electrode configurations 335 may include examples of the electrodes 315 shown and described in the wearable device 104. The electrode configuration 335-a may illustrate a bipolar electrode configuration. In the electrode configuration 335-a, an electrode 315-e may be coupled to a current generator 340 and an electrode 315-f may be coupled to ground. The current generator 340 may regulate or control a frequency of an electrical signal traveling from the electrode 315-e to the electrode 315-f. Further, the electrode configuration 335-a may include a voltage meter situated between the first electrode 315 and the second electrode 315 that is configured to measure the voltage potential between the electrode 315-e and the electrode 315-f. The electrode 315-e and the electrode 315-f may be an example of any of the combinations of electrodes (e.g., the first combination, the second combination, or the third combination) described herein.

Alternatively, the electrode configuration 335-b may illustrate a tetrapolar electrode configuration. In the electrode configuration 335-b, the wearable device 104 may utilize two electrodes (e.g., an electrode 315-g and an electrode 315-j) for driving current and two different electrodes (e.g., an electrode 315-h and an electrode 315-i) for voltage measurement. As illustrated in FIG. 3, the electrode 315-g may be coupled to the current generator 340 and an electrode 315-j may be coupled to ground resulting in voltage difference between the electrode 315-h and the electrode 315-i. Similar to the electrode configuration 335-a, the current generator 340 may regulate or control a frequency of an electrical signal traveling from the electrode 315-g to the electrode 315-j. Further, the electrode configuration 335-b may include a voltage meter situated between the electrode 315-h and the electrode 315-i which may be configured to measure a voltage potential between the electrode 315-h and the electrode 315-i. In some examples, the electrode 315-g and the electrode 315-h may be located opposite from the electrode 315-i and the electrode 315-j along the inner-housing 305-b. Alternatively, the electrode 315-g and the electrode 315-h may be located on the inner-housing 305-b and the electrode 315-i and the electrode 315-j along the outer-housing 305-a.

When a combination of electrodes 315 (e.g., the first combination, the second combination, or the third combination) is activated, the wearable device 104 may generate an electrical signal using a first electrode 315 (e.g., the electrode 315-a, the electrode 315-b, the electrode 315-c, or the electrode 315-d acting as the anode) and the electrical signal may travel along the signal path 320 (e.g., using any of the signal paths 320) through different layers of the user's skin to a second electrode 315 (e.g., the electrode 315-a, the electrode 315-b, the electrode 315-c, or the electrode 315-d acting as a cathode).

As one example, the electrode 315-b may generate the electrical signal and the electrode 315-a may receive the electrical signal from the electrode 315-b. In this example, the electrical signal may travel through all the layers of the user's finger including blood vessels of the user's finger. The wearable device 104 may compare the electrical signal generated using the electrode 315-b and the electrical signal received using the electrode 315-a and determine a bioimpedance value for the user using the comparison. As an example, the wearable device 104 may calculate a resistivity of the electrical signal using, for example, a voltage (e.g., measured by the voltage meter) and a current associated with the electrical signal (e.g., regulated by the current generator). In some aspects, techniques described herein may implement bioelectrical impedance analysis (BIA) in which the current (e.g., electrical signal) may be split into different components such as an in-phase component (I), an out-of-phase component (Q), and a magnitude (Mag, or a vector sum of both the in-phase component and the out-of-phase component). Compared to the out-of-phase component, the in-phase component may be more susceptible to a total body water of the user. In this regard, the analysis of an electrical signal received by an electrode 315 may be based on the resistance of the body, and also sub-components of the electrical current. By breaking down a received current/electrical signal into the respective components (e.g., I, Q, and Mag), the properties of the respective components may be analyzed to calculate an estimation of body composition.

The wearable device 104 may then use the resistivity or other metrics (e.g., a distance between the two electrodes, a cross-sectional area of the ring or finger, one or more components of the current, among other metrics) to determine a bioimpedance value for the user. In this example, by measuring resistivity/bioimpedance through the user's finger, the wearable device 104 may be able to determine data associated with the user's blood flowing through blood vessels in the finger, such as fat content in the user's blood.

In another example, the wearable device 104 may sweep through different frequencies in order to determine bioimpedance values for the user. In other words, the wearable device 104 may be configured to sweep through different frequencies of electrical signals to determine what frequency should be used for performing different types of bioimpedance measurements (e.g., first frequency for measuring fat content in blood, second frequency for measuring body composition).

For example, the wearable device 104 may generate a set of reference electrical signals associated with a set of different frequencies. In this example, the wearable device 104 may compare the set of reference signals received at an electrode 315 to determine which reference signal (and therefore which frequency) is most effective at highlighting changing body composition parameters. The wearable device 104 may then utilize the determined frequency for future bioimpedance measurements.

By way of another example, using a first electrode 315 (a current generator coupled to the first electrode 315), the wearable device 104 may generate a first electrical signal associated with a first frequency. Further, the wearable device 104 may generate a second electrical signal associated with a second frequency. The wearable device 104 may receive the first electrical signal and the second electrical signal using a second electrode 315 and utilize a comparison of the first electrical signal and the second electrical signal to determine a bioimpedance value, and to determine which of the first or second frequencies should be used for performing future bioimpedance measurements. As one example, the wearable device 104 may determine a shift between the first electrical signal and the second electrical signal and utilize the frequency shift to determine the bioimpedance value for the user.

In some examples, the bioimpedance value for the user may be a dynamic value. That is, the value of bioimpedance for the user may change over time or in accordance to different situations. For example, when a user eats a meal high in fat (e.g., fat content of the blood increases), the bioimpedance value for the user may increase when compared to a baseline bioimpedance value for the user. The bioimpedance value may increase because when the user eats, a fat content of the food may be packaged in the blood (e.g., glycoproteins) which changes the composition of the blood. To determine whether the user has consumed a high fat meal or that fat content of the blood has increased significantly (e.g., above a threshold), the wearable device 104 may implement several different techniques.

Using a first technique, the wearable device 104 may measure the bioimpedance value of the user according to some periodicity. As an example, the wearable device 104 may measure the bioimpedance of the user every 15 minutes. The wearable device 104 may determine that the user has consumed a high fat meal or a blood glucose level of the user has increased significantly if a difference between a bioimpedance value and a previous bioimpedance value satisfies a threshold. In some cases, different types of bioimpedance measurements (e.g., blood fat content, body composition measurements) may be performed in accordance with differing periodicities. Additionally, or alternatively, the wearable device 104 and/or corresponding system may evaluate trends associated with the user's bioimpedance measurements, and may therefore use bioimpedance measurements to evaluate the user's longer-term nutrition habits. As such, techniques described herein may utilize bioimpedance measurements in a more holistic manner to evaluate the user's long-term nutrition, eating habits, and overall health.

In some aspects, the respective bioimpedance thresholds that are used herein to evaluate bioimpedance measurements may include general bioimpedance thresholds that are known to be associated with “normal” or “healthy” humans, user-specific thresholds (e.g., thresholds based on prior bioimpedance measurements collected from the user), or both.

Using a second technique, the wearable device 104 may perform periodic baseline bioimpedance measurements to establish a baseline bioimpedance value for the user. For example, the wearable device 104 may perform a baseline bioimpedance measurement every 24 hours. In another example, the wearable device 104 may determine an occasion (e.g., a time of day) to perform the baseline bioimpedance measurement using measurement data from other sensors of the wearable device 104. For example, the wearable device 104 may utilize sleep metrics as described in FIG. 2 to determine when to perform the baseline bioimpedance measurement. As an example, the wearable device 104 may perform the baseline bioimpedance measurement during REM sleep.

Further, the wearable device 104 may determine an aperiodic bioimpedance measurement upon detecting one or more triggers (e.g., satisfaction of one or more trigger conditions). The one or more triggers may include gesture detection (e.g., detecting a repeating motion of raising and lowering the hand including the wearable device 104, which may be indicative of eating or drinking), a decrease in a temperature of the finger, a decrease in heart rate of the user, etc. Upon detecting the one or more triggers, the wearable device 104 may determine the bioimpedance value of the user and compare the bioimpedance value to the most recent baseline bioimpedance value. If a difference between the bioimpedance value and the baseline bioimpedance value exceeds a threshold, the wearable device 104 may determine that a fat content of the blood has increased significantly which may be indicative that the user has consumed a high fat meal. In some examples, the wearable device 104 may delay measuring the bioimpedance value for the user after detecting the one or more triggers. As an example, the wearable device 104 may delay the bioimpedance measurement 15, 30, or 60 minutes after detecting the one or more triggers (e.g., 60 minutes after detecting gestures that suggest the user drank a beverage or consumed a meal).

In another example, the wearable device 104 may determine changes in a body composition of the user. Similar to when the user eats a high fat meal, as the user gains fat, bioimpedance measurements for the user may increase. To determine a change in body composition, the wearable device 104 may determine bioimpedance measurements according to some periodicity. As one example, the periodicity may be equal to 24 hours. At each periodic time interval, the wearable device 104 may prompt the user to perform the bioimpedance measurement (e.g., using the second combination of electrodes 315 or the third combination of electrodes 315) such that the wearable device 104 may determine the bioimpedance values of the user at each periodic time interval. To perform the bioimpedance measurement, the user may touch a finger to an electrode 315 located at or within the outer-housing 305-a (e.g., the electrode 315-c and the electrode 315-d). In some examples, the wearable device 104 may store the bioimpedance values taken at each periodic time interval in a memory device of the wearable device 104. Because a change in the user's body composition from day-to-day may be small, the wearable device 104 may track the body composition of the user over a long time period (e.g., several days or months). If a slope of the bioimpedance values over the time period exceeds a threshold or if a difference between two or more bioimpedance values over the time period exceeds a threshold, the wearable device 104 may determine that the user is gaining fat, and may provide messages or other guidance to the user via the GUI of the user device 106.

In some examples, other factors (e.g., different from fat) may affect the body composition measurements of the user. For example, muscle as well as fat may increase bioimpedance measurements. As such, the wearable device 104 may take into account a muscle mass of the user when calculating the bioimpedance value of the user. As an example, the system may utilize motion/acceleration data collected by the wearable device 104 to determine whether the user is training or not. If the user is training, this may indicate that the user is gaining muscle and the wearable device 104 may account for the muscle gain in the bioimpedance measurement (e.g., by lowering the measured bioimpedance value by some amount).

In addition to fat concentration and body composition, other physiological and/or body composition measurements may be made using bioimpedance values. For example, using bioimpedance values, the wearable device 104 may measure changes in a hydration level, blood pressure, heart rate, or breathing of the user. Further, the wearable device 104 may determine whether there is good skin contact between the ring and the finger (e.g., excessive sweating may cause higher bioimpedance values). Moreover, in some cases, bioimpedance measurements may be used to evaluate a user's overall nutrition habits over time, how well the user is recovering from exercise or illness, etc. Further, in some cases, the system may generate alerts or messages when significant changes in the user's bioimpedance measurements are identified (e.g., when a change relative to the user's baseline bioimpedance data exceeds some threshold).

In some examples, the wearable device 104 may utilize other measurements or signals to calibrate or validate the bioimpedance value. For example, if the wearable device 104 determines an increase in bioimpedance, the wearable device may then check other metrics to confirm that the increase in bioimpedance is due to an increase in a fat content of the blood of the user. The other metrics may include a temperature metric or a HRV metric. If one or both of the temperature metric or the HRV is also indicative of an increase in a fat content of the blood (e.g., temperate value and heart rate are decreasing), the wearable device 104 may validate the increase in bioimpedance is due to an increase in a fat content of the blood of the user. Further, the wearable device 104 may validate the bioimpedance value by evaluating a signal quality of physiological signals. For example, if a signal quality (e.g., signal strength) of a PPG signal (as described in FIG. 2) is above a threshold, the wearable device 104 may validate bioimpedance value. That is, the signal strength of the PPG signal may be indicative of a signal strength of the electrical signal and therefore, the accuracy of the bioimpedance measurement.

In some examples, different frequencies of electrical current may be more susceptible to changes in a fat content of the blood and body composition changes of the user. For example, an electrical current with a first frequency (e.g., frequency within the kilohertz range) may be more susceptible to changes in a fat content of the blood and an electrical current with a second frequency (e.g., frequency within the megahertz range) may be more susceptible to body composition changes. As such, the wearable device 104 may employ different frequencies for different biological measurements being made. As such, sweeping through various frequencies used for bioimpedance measurements, as described herein, may enable the wearable device to identify different frequencies that will be used for different bioimpedance measurements.

As an example, the wearable device 104 may determine bioimpedance values for the purpose of measuring a fat content of the blood should be determined based on an electrical signal associated with the first frequency. That is, the wearable device 104 may select different frequencies for different biological measurements made using bioimpedance. In another example, the wearable device 104 may sweep through a set of frequencies to determine which frequency is the most susceptible to bioimpedance changes, and utilize the determined frequency for future biological measurements that utilize bioimpedance values.

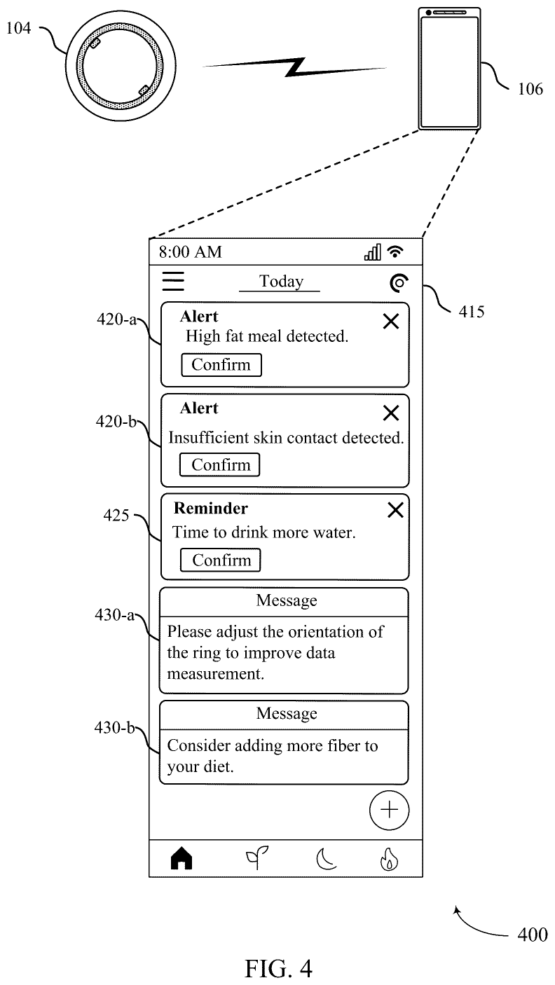

In some examples, upon detecting changes in the bioimpedance value of the user, the wearable device 104 may provide the user with a message including instructions or an alert related to the bioimpedance values of the user. In some examples, the wearable device 104 may be in communication with a user device 106 (e.g., a cellphone, a tablet, laptop, etc.) and cause a GUI of the user device 106 to display the message or the alert. Aspects related to such messages are described with more detail in FIG. 4.

FIG. 4 illustrates an example of a GUI 400 that supports techniques for collecting bioimpedance data using a wearable device in accordance with aspects of the present disclosure. The GUI 400 may implement, or be implemented by, aspects of a system 100, a system 200, and a system 300. For example, the GUI 400 may be an example of a GUI 275 of a user device 106 corresponding to a user 102 as described with reference to FIG. 2. In some examples, the GUI 400 may illustrate a series of application pages 415 which may be displayed to a user via the GUI 400 (e.g., GUI 275 illustrated in FIG. 2).

As described with reference to FIG. 2, a user may be associated with the wearable device 104 and a user device 106. In some examples, the wearable device 104 and the user device 106 may establish a communication link and communicate with one another using the communication link. In some examples, the wearable device 104 may send processed data to the user device 106. For example, the wearable device 104 may transmit bioimpedance data to the user device 106. An example of the bioimpedance data may be a difference between two bioimpedance values measured by the wearable device 104 as described with reference to FIG. 3. The bioimpedance data may be indicative of a change in a biological parameter associated with the user. For example, the bioimpedance data may indicate a change in body composition, blood content, blood pressure, glucose, hydration, heart rate, breathing rate (e.g., respiration rate), etc. The wearable device 104 may determine the bioimpedance data using one or more of the methods as described with reference to FIG. 3.

Upon receiving the bioimpedance data, the user device 106 may generate one or more one or more messages 430, one or more alerts 420, and one or more reminders 425 for display on the GUI 400. To access the messages 430, the alerts 420, and the reminders 425, the user may open an application (e.g., wearable application 250 illustrated in FIG. 2) associated with the wearable device 104. An application page 415 (e.g., an application homepage) of the application may display the one or more messages 430, the one or more alerts 420, or the one or more reminders 425 associated with the bioimpedance data.

As described with reference to FIG. 2, the bioimpedance data may indicate that the user consumed a high fat meal. In such examples, the application page 415 may display an alert 420-a. The alert 420-a may include text that reads “high fat meal detected.” Further, the application page 415 may display a message 430-b. The message 430-b may include text that reads “consider adding more fiber to diet.” In some examples, the application page 415 may display the message 430-b after the wearable device 104 detects that the bioimpedance data repeatedly indicates the user has consumed a high fat meal (e.g., the user has consumed a number of high fat meals that exceeds a threshold).

As another example, the bioimpedance data may indicate that a combination of electrodes used to determine the bioimpedance data are not in contact with the user's skin (e.g., collected bioimpedance measurements are outside a range of typical/baseline bioimpedance measurements for the user). In such examples, the application page 415 may display an alert 420-b. The alert 420-b may include text that reads “insufficient skin contact detected.” Further, the display may include message 430-a. The message 430-a may read “please adjust the orientation of the ring to improve data measurement.” Additionally, or alternatively, the bioimpedance data may indicate that the user is dehydrated. In such examples, the application page 415 may display reminder 425. The reminder 425 may read “time to drink more water.” Using such alerts 420, messages 430, and reminders 425 may allow a user to make informed decisions on the user's health.

In some implementations, the user may be able to trigger bioimpedance measurements (e.g., body composition measurements) via the application page 415. For example, through the application page 415, the user may generate a user input that causes the wearable device 104 to selectively activate electrodes of the wearable device to perform bioimpedance measurements. In some cases, the application page 415 may display prompts for performing bioimpedance measurements. As described previously herein, the system may prompt the user to perform bioimpedance measurements at regular or irregular intervals (e.g., an hour before bed every day), based on detected triggers (e.g., based on identifying eating or drinking-related gestures), and the like. In some aspects, upon triggering a bioimpedance measurement, the application page 415 may display a set of instructions that instruct the user on how to perform the bioimpedance measurements (e.g., instruct the user to place a finger of their non-ring-wearing hand on an electrode on the outer circumferential surface of the ring 104).

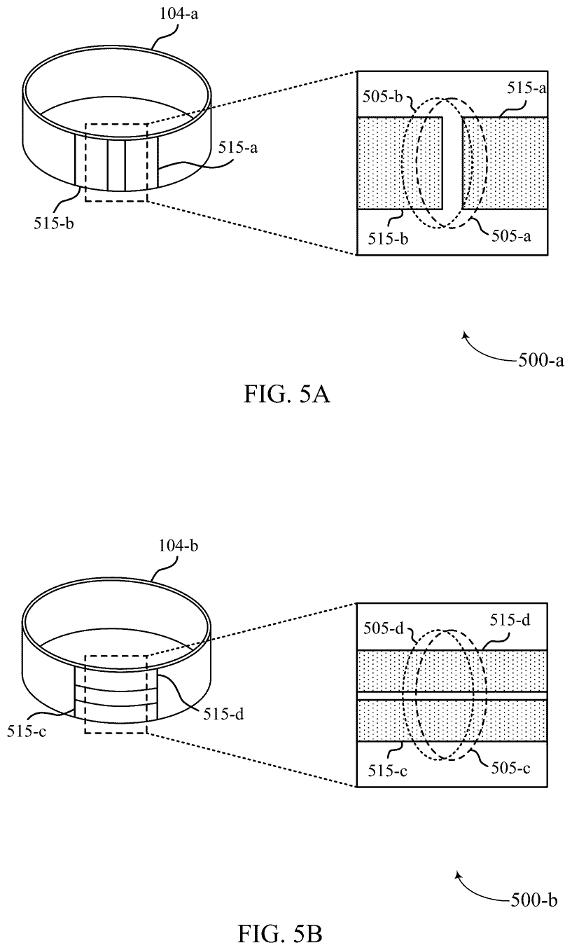

FIGS. 5A and 5B illustrate examples of electrode configurations 500 (e.g., an electrode configuration 500-a and an electrode configuration 500-b) that support techniques for collecting bioimpedance data using a wearable device in accordance with aspects of the present disclosure. In some examples, the electrode configurations 500 may implement aspects of the system 100, the system 200, the system 300, the GUI 400, or any combination thereof. For example, the electrode configurations 500 may include one or more electrodes 515 (e.g., an electrode 515-a, an electrode 515-b, an electrode 515-c, or an electrode 515-d) which may be examples of electrodes 315 as described with reference to FIG. 3.

As described with reference to FIG. 3, wearable devices 104 (e.g., a wearable device 104-a and a wearable device 104-b) may utilize electrodes 515 to perform bioimpedance measurements for users of the wearable devices 104. In FIGS. 5A and 5B, two possible electrode configurations 500 for electrodes 515 are illustrated.

The electrode configuration 500-a illustrates an “in-line” electrode design. In the electrode configuration 500-a, the wearable device 104-a may include at least two electrodes 515. For example, the wearable device 104-a may include an electrode 515-a and an electrode 515-b. As shown in FIG. 5A, the electrode 515-a and the electrode 515-b may be located on an outside surface of the wearable device 104-a (e.g., disposed within an outer housing of the wearable device 104-a). In additional or alternative embodiments, the electrodes 515-a, 515-b of the in-line design may be implemented within the inner circumferential surface of the wearable device 104-a (e.g., disposed within an inner housing of the wearable device 104-a).

FIG. 5A illustrates the electrodes 515 as square shaped or rectangular shaped. However, other shapes are possible (e.g., circular shaped electrodes or oval shaped electrodes). Further, as illustrated in FIG. 5A, the electrode 515-a and the electrode 515-b may span at least a portion of a width of the band of the wearable device 104-a and may be parallel to one another in a vertical direction. In some examples, a size of the electrode 515-a and the electrode 515-b may be small enough such that a finger of the user may cover the entirety of the electrode 515-a and/or the electrode 515-b during a bioimpedance measurement. In such cases, the amount of contact between the user's tissue (e.g., finger) and the electrodes 515 may remain constant across/between bioimpedance measurements (because the user's tissue contacts the entirety of the electrodes 515). Additionally, the electrodes 515 may not overlap one another and there may be a space between the electrodes 515 that separate them from one another.

As described with reference to FIG. 3, a user of the wearable device 104-a may touch a surface of one or both electrodes 515 to perform the bioimpedance measurements. As an example, the user may place a finger at a location 505-a or a location 505-b such that the user's finger is in contact with at least one of the electrodes 515. For instance, the first electrode 515-a may transmit an electrical signal that travels through the user's tissue, and is received by the second electrode 515-b (or vice versa). In additional or alternative implementations, as described previously herein with respect to FIG. 3, the electrodes 515-a, 515-b may be used in combination with electrodes on the inner circumferential surface of the wearable device 104-a. For example, electrodes on the inner surface of the wearable device 104-a may transmit an electrical signal that travels up through the user's arm, across their chest, and down the other arm to a finger contacting the electrode(s) 515-a, 515-b so that the electrical signal is received by the electrode(s) 515-a, 515-b (or vice versa, where the electrodes 515-a, 515-b are the transmitting electrodes, and electrodes on the inner surface are the receiving electrodes).

In some examples, the wearable device 104-a may perform multiple bioimpedance measurements. When performing multiple bioimpedance measurements, the user may not place their finger on the electrodes 515 in an exact same location 505 for each of the multiple bioimpedance measurements. For example, while performing a first bioimpedance measurement at a first time, the user may place their finger at the location 505-a and, during a second bioimpedance measurement and at a second time, the user may place their finger at the location 505-b. Moving the finger from location 505-a to location 505-b may change the amount of surface area that the user's finger contacts the respective electrodes 515-a, 515-b. For example, when in location 505-a, the user's finger may contact approximately equal amounts of surface area within the electrodes 515-a, 515-b. Comparatively, when in location 505-b, the user's finger may contact more surface area within the electrode 515-b as compared to the surface area contacts within the electrode 515-a. Such changing surface area contacts may result in variance in bioimpedance measurements performed by the wearable device 104. That is, bioimpedance measurements may vary based on the location 505 of the user's finger (e.g., based on the quantity and/or proportions of surface area contacted within the respective electrodes).

As an alternative, the electrode configuration 500-b illustrates a parallel or “striped” electrode design. In the electrode configuration 500-b, the wearable device 104-b may include at least two electrodes 515. For example, the wearable device 104-b may include an electrode 515-c and an electrode 515-d. As shown in FIG. 5B, the electrode 515-c and the electrode 515-d may be located on an outside surface of the wearable device 104-b (or may be disposed within the outer housing of the wearable device 104-b). In additional or alternative embodiments, the electrodes 515-c, 515-d of the parallel design may be implemented within the inner circumferential surface of the wearable device 104-b (e.g., disposed within an inner housing of the wearable device 104-b).

FIG. 5B illustrates the electrodes 515 as square shaped or rectangular shaped. However, other shapes are possible (e.g., circular shaped electrodes or oval shaped electrodes). Further, as illustrated in FIG. 5B, each electrode 515 may run along at least a portion of a length of the band of the wearable device 104-b and may be parallel to one another in a horizontal direction. In some examples, a size of the electrode 515-c and the electrode 515-d may be small enough such that a finger of the user may cover the entirety of the electrode 515-c or the electrode 515-d during bioimpedance measurements. In such cases, the amount of contact between the user's tissue (e.g., finger) and the electrodes 515 may remain constant across/between bioimpedance measurements (because the user's tissue contacts the entirety of the electrodes 515). Additionally, the electrodes 515 may not overlap one another and there may be a space between the electrodes 515 that separate them from one another.

As described with reference to FIG. 3, a user of the wearable device 104-b may touch a surface of one or both electrodes 515 to perform the bioimpedance measurements. As an example, the user may place a finger at a location 505-c or a location 505-d such that the user's finger is in contact with at least one of the electrodes 515. Further, in some examples, the wearable device 104-b may perform multiple bioimpedance measurements. In such examples, the user may not place their finger on the electrodes 515 in an exact same location 505 for each of the multiple bioimpedance measurements.

For example, while performing a first bioimpedance measurement at a first time, the user may place their finger at the location 505-c and, during a second bioimpedance measurement and at a second time, the user may place their finger at the location 505-d. Unlike the electrode configuration 500-a, moving the finger from the location 505-c to the location 505-d may not change the amount of surface area that the finger and an electrode 515 are in contact. In other words, the electrode configuration 500-b may reduce (or eliminate) noise/variance in bioimpedance measurements that results from changing contact locations and surface areas between the electrodes 515 and the user's tissue. For example, the finger of the user may cover a similar or same amount of surface area of the electrodes 515-c, 515-d at the location 505-b when compared to the location 505-a.

Although the electrode configurations 500 illustrate two electrodes 515 disposed on an outer housing of the wearable device 104, the electrode configurations 500 may be applied to electrodes 515 disposed on different areas of the wearable device 104. For example, the electrode configurations 500 may apply to electrodes 515 disposed on an inner-housing of the wearable device 104.

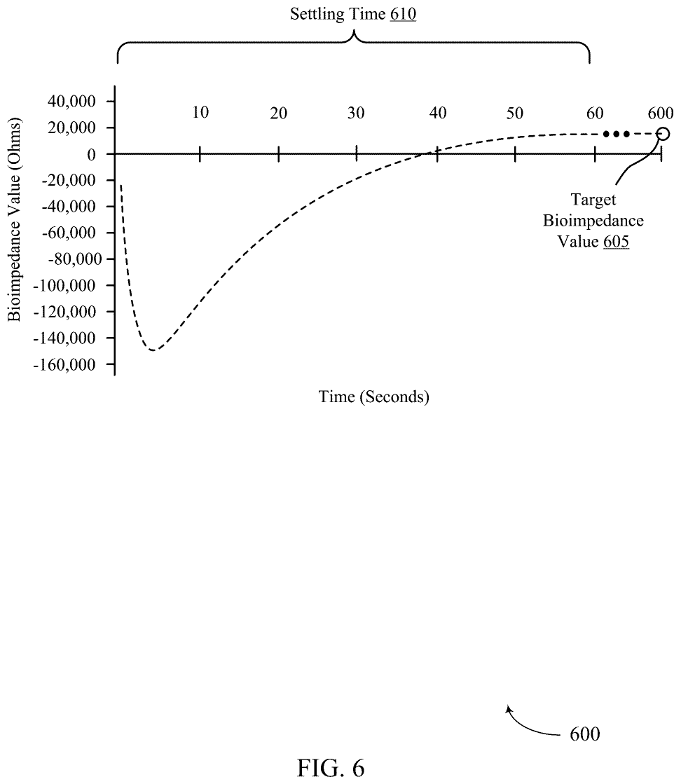

FIG. 6 illustrates an example of a bioimpedance-time diagram 600 that supports techniques for collecting bioimpedance data using a wearable device in accordance with aspects of the present disclosure. In some examples, the bioimpedance-time diagram 600 may be implemented by aspects of a system 100, a system 200, a system 300, a GUI 400, or electrode configurations 500.

As described with reference to FIG. 3, a wearable device may utilize electrodes to determine a bioimpedance value for a user of the wearable device. As shown in FIG. 6, after applying a voltage to the Tx electrodes (e.g., generating an electrical signal), and subsequently receiving the electrical signal with the Rx electrodes, a bioimpedance value for the user may take time to stabilize or settle. For example, as shown in FIG. 6, the bioimpedance value may begin to stabilize or settle at 600 seconds or 10 minutes from when the wearable device applies the voltage to the electrodes (e.g., transmit and receive electrical signals). The time it takes for the bioimpedance to stabilize or settle may be known as a “settling time 610.” In other words, the settling time 610 may indicate an amount of time for the voltage applied to the electrodes (e.g., the electrical signals transmitted/received by the electrodes) to stabilize to a relatively constant value. In some cases, the settling time may be due to material changes and breaks in contact between the user's tissue and the electrodes. As such, in clinical settings, the settling time 610 shown in FIG. 6 may be reduced or eliminated using a conductive gel or a liquid between the skin of the user and the electrodes. However, conductive gels may not be feasible or practical in the context of a wearable device 104, and the “dry electrodes” of the wearable device may experience the settling time 610 for bioimpedance measurements.

In some cases, to account for the settling time 610, the wearable device 104 may be configured to perform bioimpedance measurements (e.g., transmit and receive electrical signals) for a time duration that is close to or longer than the settling time 610 (e.g., 50-60 seconds). Taking these long bioimpedance measurements may ensure that the bioimpedance values have leveled out to an accurate, reliable level (e.g., a “target bioimpedance value 605”).

However, such long measurements may be inconvenient and impractical for the user to perform on a regular basis. As such, to avoid, decrease, or otherwise account for the settling time 610, the wearable device 104 may perform collect bioimpedance data during a duration that is shorter than the settling time 610 (e.g., 15 seconds), and estimate/extrapolate the bioimpedance value for user out to/past the settling time 610 to determine/estimate the target bioimpedance value 605. In this example, the system may be configured to estimate/extrapolate the target bioimpedance value 605 that would have resulted had the bioimpedance measurements been performed for a duration that exceeded the settling time 610.

As shown in FIG. 6, the bioimpedance values, starting from approximately 5 seconds, exhibit a curve shape (e.g., exponential curve or parabolic curve). With this in mind, the wearable device 104 (and/or other components of the system, such as a user device 106, servers 110, etc.) may utilize an algorithm to analyze multiple bioimpedance values (e.g., from 10 seconds to 20 seconds) and use these bioimpedance values to determine the function of the curve. Using the algorithm, the wearable device may then estimate the target bioimpedance value 605 (or bioimpedance value at some time at or beyond the settling time 610) using the function (or find an asymptote of the curve). This may allow the wearable device to determine the bioimpedance value of the user in a shorter amount of time (e.g., shorter than 600 second or 10 minutes).

In some cases, to improve the ability of the system to estimate/extrapolate bioimpedance values, the user may perform one or more “calibration” bioimpedance measurements for a duration that is longer than the settling time 610. For example, the user may perform one or more calibration bioimpedance measurements for longer than 600 seconds (e.g., 10 minutes) such that the system can determine/estimate a duration of the settling time 610 for the wearable device 104, a shape of the curve of the bioimpedance measurements, and the like. In this regard, the system may utilize the determined duration of the settling time 610, the shape of the curve, and/or other characteristics/parameters determined based on the calibration sessions in order to generate a model or algorithm that may be used to estimate/extrapolate bioimpedance values for subsequent measurements that are not performed for the duration of the settling time 610.

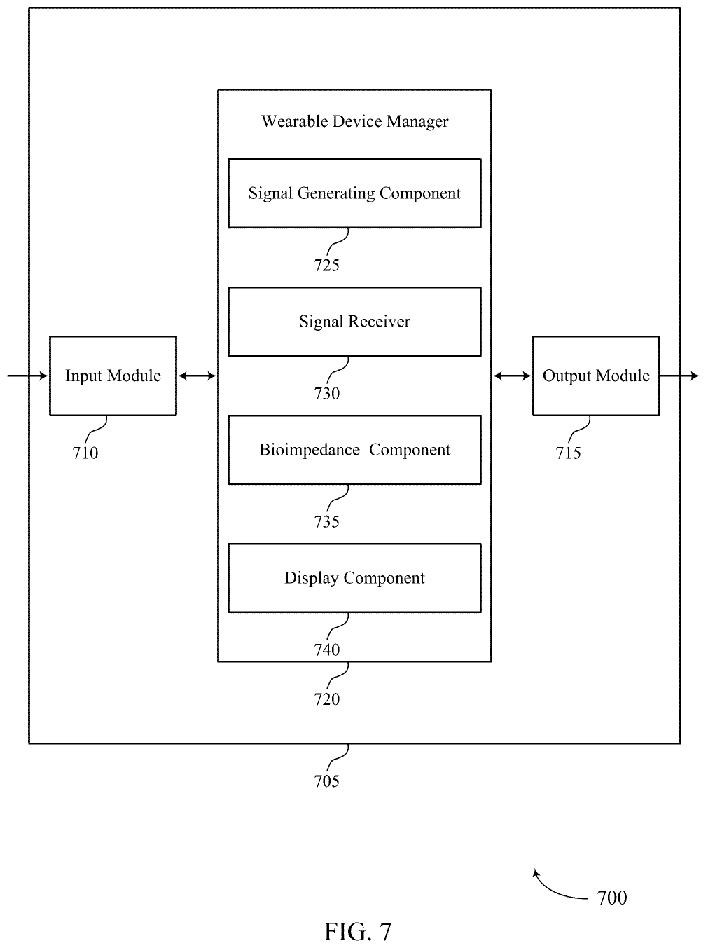

FIG. 7 illustrates a block diagram 700 of a device 705 that supports techniques for collecting bioimpedance data using a wearable device in accordance with aspects of the present disclosure. The device 705 may include an input module 710, an output module 715, and a wearable device manager 720. The device 705 may also include a processor. Each of these components may be in communication with one another (e.g., via one or more buses).

For example, the wearable device manager 720 may include a signal generating component 725, a signal receiver 730, a bioimpedance component 735, a display component 740, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The signal generating component 725 may be configured as or otherwise support a means for generating a first electrical signal using a first electrode of a wearable ring device, wherein the first electrical signal is associated with a first frequency. The signal receiver 730 may be configured as or otherwise support a means for receiving the first electrical signal using a second electrode of the wearable ring device, wherein the first electrode, the second electrode, or both, are disposed within an inner circumferential surface of the wearable ring device. The bioimpedance component 735 may be configured as or otherwise support a means for determining first bioimpedance data associated with a user based at least in part on a comparison of the first electrical signal generated by the first electrode and the first electrical signal received by the second electrode. The display component 740 may be configured as or otherwise support a means for causing a GUI of a user device to display a message associated with the first bioimpedance data.

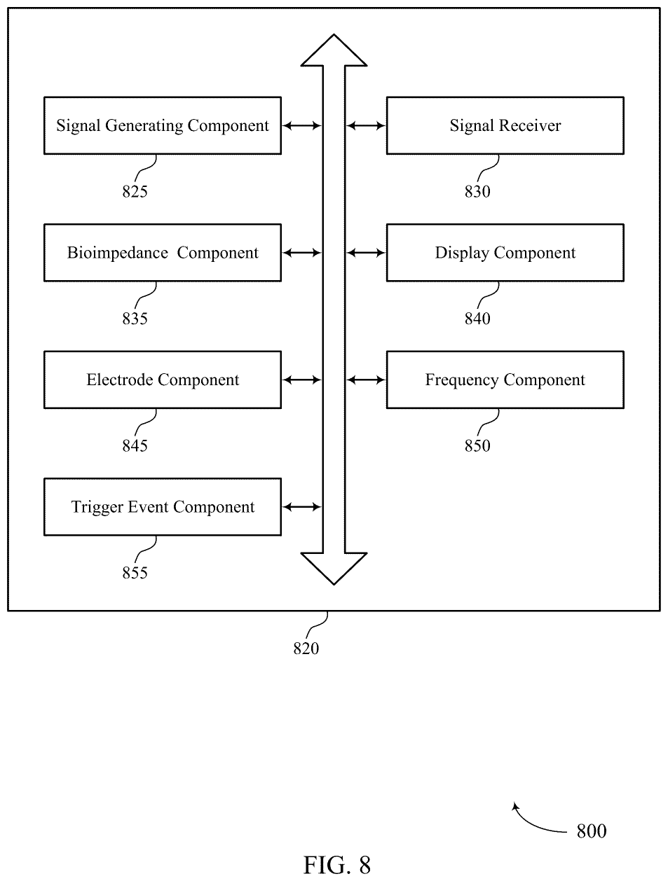

FIG. 8 illustrates a block diagram 800 of a wearable device manager 820 that supports techniques for collecting bioimpedance data using a wearable device in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of techniques for collecting bioimpedance data using a wearable device as described herein. For example, the wearable device manager 820 may include a signal generating component 825, a signal receiver 830, a bioimpedance component 835, a display component 840, an electrode component 845, a frequency component 850, a trigger event component 855, or any combination thereof. Each of these components may communicate, directly or indirectly, with one another (e.g., via one or more buses).

The signal generating component 825 may be configured as or otherwise support a means for generating a first electrical signal using a first electrode of a wearable ring device, wherein the first electrical signal is associated with a first frequency. The signal receiver 830 may be configured as or otherwise support a means for receiving the first electrical signal using a second electrode of the wearable ring device, wherein the first electrode, the second electrode, or both, are disposed within an inner circumferential surface of the wearable ring device. The bioimpedance component 835 may be configured as or otherwise support a means for determining first bioimpedance data associated with a user based at least in part on a comparison of the first electrical signal generated by the first electrode and the first electrical signal received by the second electrode. The display component 840 may be configured as or otherwise support a means for causing a GUI of a user device to display a message associated with the first bioimpedance data.

In some examples, the wearable ring device comprises a plurality of electrodes including the first electrode, and the electrode component 845 may be configured as or otherwise support a means for selectively activating the first electrode and the second electrode from the plurality of electrodes based at least in part on the first physiological parameter, wherein generating the first electrical signal, receiving the first electrical signal, or both, are based at least in part on selectively activating the first electrode and the second electrode.

In some examples, the first bioimpedance data is associated with a first biological parameter, and the signal generating component 825 may be configured as or otherwise support a means for generating a second electrical signal using the first electrode, the second electrode, or both, wherein the second electrical signal is associated with a second frequency selected based at least in part on a second biological parameter. In some examples, the first bioimpedance data is associated with a first biological parameter, and the signal receiver 830 may be configured as or otherwise support a means for receiving the second electrical signal using a third electrode that is disposed within an outer circumferential surface of the wearable ring device. In some examples, the first bioimpedance data is associated with a first biological parameter, and the bioimpedance component 835 may be configured as or otherwise support a means for determining second bioimpedance data associated with the user associated based at least in part on a comparison of the second electrical signal generated by the first electrode, the second electrode, or both, and the first electrical signal received by the third electrode. In some examples, the first bioimpedance data is associated with a first biological parameter, and the display component 840 may be configured as or otherwise support a means for causing the GUI of the user device to display a second message associated with the second biological parameter based at least in part on the second bioimpedance data.

In some examples, the first biological parameter and the second biological parameter comprise one of a blood content parameter, a body composition parameter, a blood pressure parameter, a glucose parameter, a hydration parameter, a heart rate parameter, a breathing rate parameter, or any combination thereof.

In some examples, the display component 840 may be configured as or otherwise support a means for receiving, via the GUI of the user device, a user input to perform a bioimpedance measurement, wherein generating the first electrical signal is based at least in part on the user input.

In some examples, the display component 840 may be configured as or otherwise support a means for causing the GUI of the user device to display, in response to the user input, a set of instructions that instruct the user to contact one of the first electrode or the second electrode with another portion of their body, wherein generating the first electrical signal, receiving the first electrical signal, determining the first bioimpedance data, or any combination thereof, is based at least in part on displaying the set of instructions.

In some examples, the signal generating component 825 may be configured as or otherwise support a means for generating, using the first electrode, a plurality of reference electrical signals associated with a plurality of frequencies including the first frequency. In some examples, the signal receiver 830 may be configured as or otherwise support a means for receiving the plurality of reference electrical signals using the second electrode. In some examples, the frequency component 850 may be configured as or otherwise support a means for comparing the plurality of reference electrical signals associated with the plurality of frequencies received at the second electrode. In some examples, the frequency component 850 may be configured as or otherwise support a means for selecting the first frequency from the plurality of frequencies based at least in part on the comparison, wherein generating the first electrical signal is based at least in part on selecting the first frequency.

In some examples, the trigger event component 855 may be configured as or otherwise support a means for acquiring biological data associated with the user using the wearable ring device. In some examples, the trigger event component 855 may be configured as or otherwise support a means for identifying a satisfaction of one or more trigger conditions for performing bioimpedance measurements based at least in part on the biological data, wherein generating the first electrical signal is based at least in part on the satisfaction of the one or more trigger conditions.

In some examples, the biological data comprises at least motion data, and the trigger event component 855 may be configured as or otherwise support a means for identifying one or more gestures that the user engaged in based at least in part on the motion data, wherein the satisfaction of the one or more trigger conditions is based at least in part on the one or more gestures, and wherein the one or more gestures comprise a drinking gesture, an eating gesture, or both.

In some examples, the trigger event component 855 may be configured as or otherwise support a means for determining that the user engaged in the one or more gestures at a first time. In some examples, the signal generating component 825 may be configured as or otherwise support a means for generating the first electrical signal at a second time subsequent to the first time, wherein a time duration between the first time and the second time is based at least on the one or more gestures.

In some examples, the bioimpedance component 835 may be configured as or otherwise support a means for determining baseline bioimpedance data associated with the user based at least in part on one or more additional electrical signals exchanged between the first electrode and the second electrode, wherein the one or more additional electrical signals are associated with the first frequency. In some examples, the bioimpedance component 835 may be configured as or otherwise support a means for comparing the first bioimpedance data with the baseline bioimpedance data, wherein the message is based at least in part on the comparison.

In some examples, the first electrode is disposed within the inner circumferential surface at a first radial position. In some examples, the second electrode is disposed within the inner circumferential surface at a second radial position.

In some examples, a relative timing associated with generating the first electrical signal, receiving the first electrical signal, or both, is based at least in part on a sleeping pattern associated with the user, a circadian rhythm associated with the user, or both.

In some examples, the first electrode is disposed within the inner circumferential surface of the wearable ring device. In some examples, the second electrode is disposed within an outer circumferential surface of the wearable ring device.

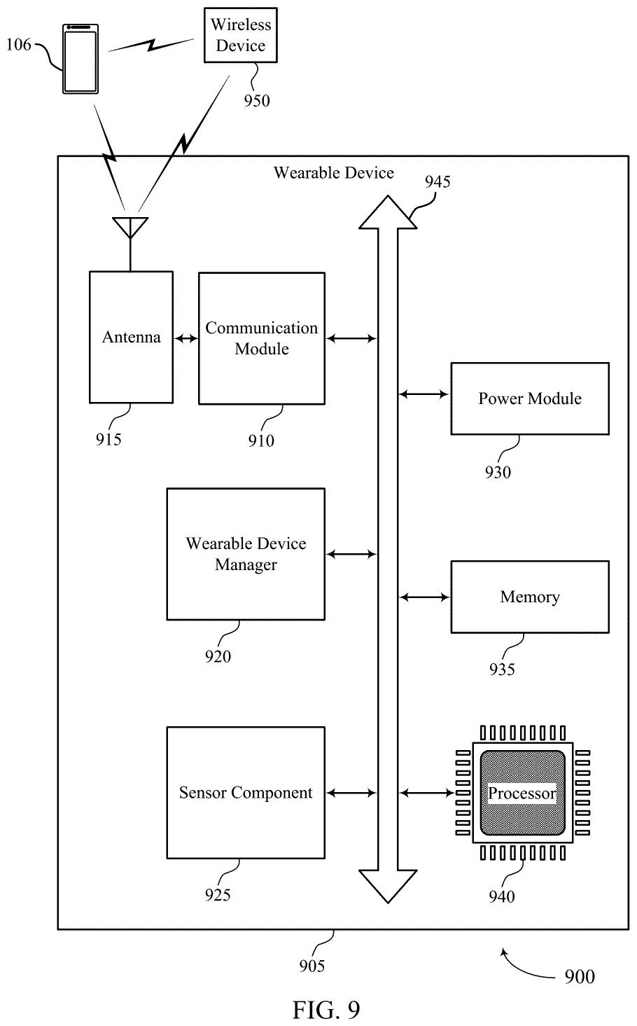

FIG. 9 illustrates a diagram of a system 900 including a device 905 that supports techniques for collecting bioimpedance data using a wearable device in accordance with aspects of the present disclosure. The device 905 may be an example of or include the components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, an antenna 915, a sensor component 925, a power module 930, a memory 935, a processor 940, and a wireless device 950. These components may be in electronic communication or otherwise coupled (e.g., operatively, communicatively, functionally, electronically, electrically) via one or more buses (e.g., a bus 945).

For example, the wearable device manager 920 may be configured as or otherwise support a means for generating a first electrical signal using a first electrode of a wearable ring device, wherein the first electrical signal is associated with a first frequency. The wearable device manager 920 may be configured as or otherwise support a means for receiving the first electrical signal using a second electrode of the wearable ring device, wherein the first electrode, the second electrode, or both, are disposed within an inner circumferential surface of the wearable ring device. The wearable device manager 920 may be configured as or otherwise support a means for determining first bioimpedance data associated with a user based at least in part on a comparison of the first electrical signal generated by the first electrode and the first electrical signal received by the second electrode. The wearable device manager 920 may be configured as or otherwise support a means for causing a GUI of a user device to display a message associated with the first bioimpedance data.

By including or configuring the wearable device manager 920 in accordance with examples as described herein, the device 905 may support techniques for determining a health metric from wearable-based bioimpedance data. In particular, the system 100 illustrated in FIG. 1 may support techniques for determining bioimpedance data health metric and causing a user device 106 corresponding to the user 102 to display a message corresponding to the bioimpedance data. The message may alert the user to potential health risks or stressors.

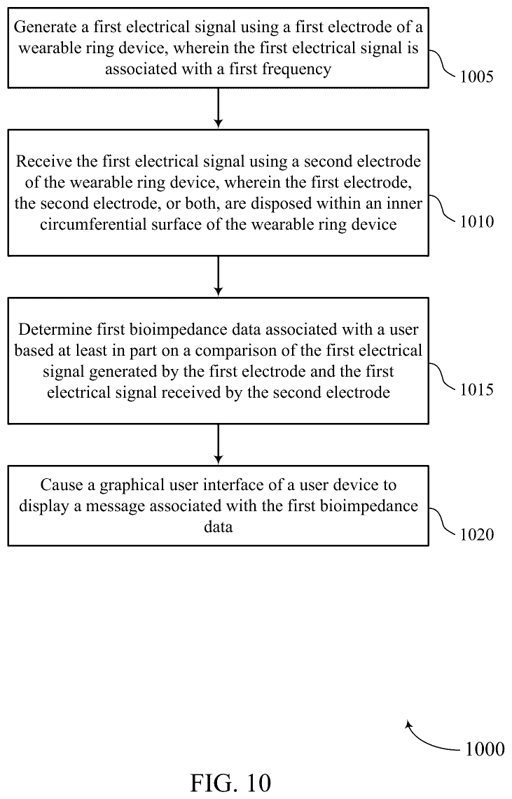

FIG. 10 illustrates a flowchart showing a method 1000 that supports techniques for collecting bioimpedance data using a wearable device in accordance with aspects of the present disclosure. The operations of the method 1000 may be implemented by a wearable device or its components as described herein. For example, the operations of the method 1000 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1005, the method may include generating a first electrical signal using a first electrode of a wearable ring device, wherein the first electrical signal is associated with a first frequency. The operations of 1005 may be performed in accordance with examples as disclosed herein. In some examples, aspects of the operations of 1005 may be performed by a signal generating component 825 as described with reference to FIG. 8.

At 1010, the method may include receiving the first electrical signal using a second electrode of the wearable ring device, wherein the first electrode, the second electrode, or both, are disposed within an inner circumferential surface of the wearable ring device. The operations of 1010 may be performed in accordance with examples as disclosed herein. In some examples, aspects of the operations of 1010 may be performed by a signal receiver 830 as described with reference to FIG. 8.

At 1015, the method may include determining first bioimpedance data associated with a user based at least in part on a comparison of the first electrical signal generated by the first electrode and the first electrical signal received by the second electrode. The operations of 1015 may be performed in accordance with examples as disclosed herein. In some examples, aspects of the operations of 1015 may be performed by a bioimpedance component 835 as described with reference to FIG. 8.

At 1020, the method may include causing a GUI of a user device to display a message associated with the first bioimpedance data. The operations of 1020 may be performed in accordance with examples as disclosed herein. In some examples, aspects of the operations of 1020 may be performed by a display component 840 as described with reference to FIG. 8.

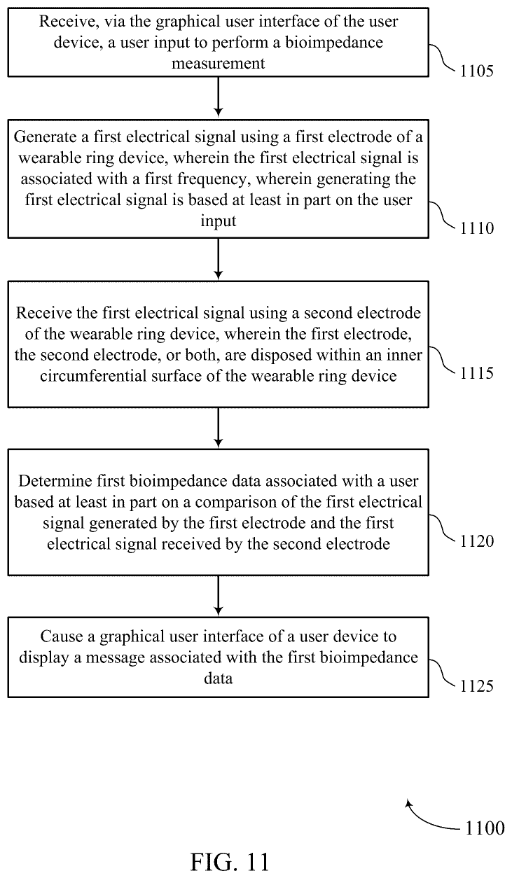

FIG. 11 illustrates a flowchart showing a method 1100 that supports techniques for collecting bioimpedance data using a wearable device in accordance with aspects of the present disclosure. The operations of the method 1100 may be implemented by a wearable device or its components as described herein. For example, the operations of the method 1100 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1105, the method may include receiving, via the GUI of the user device, a user input to perform a bioimpedance measurement. The operations of 1105 may be performed in accordance with examples as disclosed herein. In some examples, aspects of the operations of 1105 may be performed by a display component 840 as described with reference to FIG. 8.

At 1110, the method may include generating a first electrical signal using a first electrode of a wearable ring device, wherein the first electrical signal is associated with a first frequency, wherein generating the first electrical signal is based at least in part on the user input. The operations of 1110 may be performed in accordance with examples as disclosed herein. In some examples, aspects of the operations of 1110 may be performed by a signal generating component 825 as described with reference to FIG. 8.

At 1115, the method may include receiving the first electrical signal using a second electrode of the wearable ring device, wherein the first electrode, the second electrode, or both, are disposed within an inner circumferential surface of the wearable ring device. The operations of 1115 may be performed in accordance with examples as disclosed herein. In some examples, aspects of the operations of 1115 may be performed by a signal receiver 830 as described with reference to FIG. 8.

At 1120, the method may include determining first bioimpedance data associated with a user based at least in part on a comparison of the first electrical signal generated by the first electrode and the first electrical signal received by the second electrode. The operations of 1120 may be performed in accordance with examples as disclosed herein. In some examples, aspects of the operations of 1120 may be performed by a bioimpedance component 835 as described with reference to FIG. 8.

At 1125, the method may include causing a GUI of a user device to display a message associated with the first bioimpedance data. The operations of 1125 may be performed in accordance with examples as disclosed herein. In some examples, aspects of the operations of 1125 may be performed by a display component 840 as described with reference to FIG. 8.

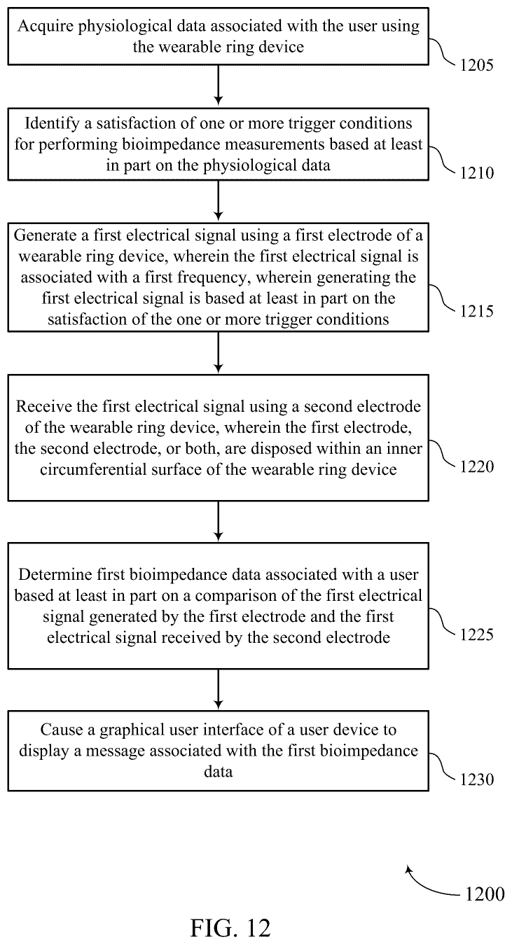

FIG. 12 illustrates a flowchart showing a method 1200 that supports techniques for collecting bioimpedance data using a wearable device in accordance with aspects of the present disclosure. The operations of the method 1200 may be implemented by a wearable device or its components as described herein. For example, the operations of the method 1200 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1205, the method may include acquiring biological data associated with the user using the wearable ring device. The operations of 1205 may be performed in accordance with examples as disclosed herein. In some examples, aspects of the operations of 1205 may be performed by a trigger event component 855 as described with reference to FIG. 8.

At 1210, the method may include identifying a satisfaction of one or more trigger conditions for performing bioimpedance measurements based at least in part on the biological data. The operations of 1210 may be performed in accordance with examples as disclosed herein. In some examples, aspects of the operations of 1210 may be performed by a trigger event component 855 as described with reference to FIG. 8.

At 1215, the method may include generating a first electrical signal using a first electrode of a wearable ring device, wherein the first electrical signal is associated with a first frequency, wherein generating the first electrical signal is based at least in part on the satisfaction of the one or more trigger conditions. The operations of 1215 may be performed in accordance with examples as disclosed herein. In some examples, aspects of the operations of 1215 may be performed by a signal generating component 825 as described with reference to FIG. 8.

At 1220, the method may include receiving the first electrical signal using a second electrode of the wearable ring device, wherein the first electrode, the second electrode, or both, are disposed within an inner circumferential surface of the wearable ring device. The operations of 1220 may be performed in accordance with examples as disclosed herein. In some examples, aspects of the operations of 1220 may be performed by a signal receiver 830 as described with reference to FIG. 8.

At 1225, the method may include determining first bioimpedance data associated with a user based at least in part on a comparison of the first electrical signal generated by the first electrode and the first electrical signal received by the second electrode. The operations of 1225 may be performed in accordance with examples as disclosed herein. In some examples, aspects of the operations of 1225 may be performed by a bioimpedance component 835 as described with reference to FIG. 8.

At 1230, the method may include causing a GUI of a user device to display a message associated with the first bioimpedance data. The operations of 1230 may be performed in accordance with examples as disclosed herein. In some examples, aspects of the operations of 1230 may be performed by a display component 840 as described with reference to FIG. 8.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary.” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
generating a first electrical signal using a first electrode of a wearable ring device, wherein the first electrical signal is associated with a first frequency;
receiving the first electrical signal using a second electrode of the wearable ring device, wherein the first electrode, the second electrode, or both, are disposed within an inner circumferential surface of the wearable ring device;
determining first bioimpedance data associated with a user based at least in part on a comparison of the first electrical signal generated by the first electrode and the first electrical signal received by the second electrode; and
causing a graphical user interface of a user device to display a message associated with the first bioimpedance data.

2. The method of claim 1, wherein the wearable ring device comprises a plurality of electrodes including the first electrode, the second electrode, and a third electrode, and wherein the first bioimpedance data is associated with a first biological parameter of a plurality of biological parameters, the method further comprising:
selectively activating the first electrode and the second electrode from the plurality of electrodes based at least in part on the first biological parameter, wherein generating the first electrical signal, receiving the first electrical signal, or both, are based at least in part on selectively activating the first electrode and the second electrode.

3. The method of claim 1, wherein the first bioimpedance data is associated with a first biological parameter, and wherein the first frequency is selected based at least in part on the first biological parameter, the method further comprising:
generating a second electrical signal using the first electrode, the second electrode, or both, wherein the second electrical signal is associated with a second frequency selected based at least in part on a second biological parameter;
receiving the second electrical signal using a third electrode that is disposed within an outer circumferential surface of the wearable ring device;
determining second bioimpedance data associated with the user associated based at least in part on a comparison of the second electrical signal generated by the first electrode, the second electrode, or both, and the first electrical signal received by the third electrode; and
causing the graphical user interface of the user device to display a second message associated with the second biological parameter based at least in part on the second bioimpedance data.

4. The method of claim 3, wherein the first biological parameter and the second biological parameter comprise one of a blood content parameter, a body composition parameter, a blood pressure parameter, a glucose parameter, a hydration parameter, a heart rate parameter, a breathing rate parameter, or any combination thereof.

5. The method of claim 1, further comprising:
receiving, via the graphical user interface of the user device, a user input to perform a bioimpedance measurement, wherein generating the first electrical signal is based at least in part on the user input.

6. The method of claim 5, further comprising:
causing the graphical user interface of the user device to display, in response to the user input, a set of instructions that instruct the user to contact one of the first electrode or the second electrode with another portion of their body, wherein generating the first electrical signal, receiving the first electrical signal, determining the first bioimpedance data, or any combination thereof, is based at least in part on displaying the set of instructions.

7. The method of claim 1, further comprising:
generating, using the first electrode, a plurality of reference electrical signals associated with a plurality of frequencies including the first frequency;
receiving the plurality of reference electrical signals using the second electrode;
comparing the plurality of reference electrical signals associated with the plurality of frequencies received at the second electrode; and
selecting the first frequency from the plurality of frequencies based at least in part on the comparison, wherein generating the first electrical signal is based at least in part on selecting the first frequency.

8. The method of claim 1, further comprising:
acquiring biological data associated with the user using the wearable ring device; and
identifying a satisfaction of one or more trigger conditions for performing bioimpedance measurements based at least in part on the biological data, wherein generating the first electrical signal is based at least in part on the satisfaction of the one or more trigger conditions.

9. The method of claim 8, wherein the biological data comprises at least motion data, the method further comprising:
identifying one or more gestures that the user engaged in based at least in part on the motion data, wherein the satisfaction of the one or more trigger conditions is based at least in part on the one or more gestures, and wherein the one or more gestures comprise a drinking gesture, an eating gesture, or both.

10. The method of claim 9, further comprising:
determining that the user engaged in the one or more gestures at a first time; and
generating the first electrical signal at a second time subsequent to the first time, wherein a time duration between the first time and the second time is based at least on the one or more gestures.

11. The method of claim 1, further comprising:
determining baseline bioimpedance data associated with the user based at least in part on one or more additional electrical signals exchanged between the first electrode and the second electrode, wherein the one or more additional electrical signals are associated with the first frequency; and
comparing the first bioimpedance data with the baseline bioimpedance data, wherein the message is based at least in part on the comparison.

12. The method of claim 1, wherein the first electrode is disposed within the inner circumferential surface at a first radial position, and wherein the second electrode is disposed within the inner circumferential surface at a second radial position.

13. The method of claim 1, wherein a relative timing associated with generating the first electrical signal, receiving the first electrical signal, or both, is based at least in part on a sleeping pattern associated with the user, a circadian rhythm associated with the user, or both.

14. The method of claim 1, wherein the first electrode is disposed within the inner circumferential surface of the wearable ring device, and wherein the second electrode is disposed within an outer circumferential surface of the wearable ring device.

15. The method of claim 1, wherein the first electrode and the second electrode are disposed within a same surface of the wearable ring device, wherein the first electrode and the second electrode are parallel to one another and extend across a same angular distance around a circumference of the surface, wherein the first electrode and the second electrode are associated with a same height and a same length.

16. The method of claim 1, wherein the first electrical signal is transmitted by the first electrode and received by the second electrode for a time duration, wherein determining the first bioimpedance data comprises:
determining a change in bioimpedance values throughout the time duration based at least in part on the first electrical signal received by the second electrode throughout the time duration;
estimating a settling time for the bioimpedance values; and
extrapolating the bioimpedance values to the settling time based at least in part on the time duration being less than the settling time, wherein the first bioimpedance data is determined based at least in part on the extrapolating.

17. A wearable ring device, comprising:
a first electrode;
a second electrode, wherein the first electrode, the second electrode, or both, are disposed within an inner circumferential surface of the wearable ring device; and
a controller communicatively coupled to the first electrode and the second electrode, the controller configured to:
generate a first electrical signal using the first electrode of the wearable ring device, wherein the first electrical signal is associated with a first frequency;
receive the first electrical signal using the second electrode of the wearable ring device;
determine first bioimpedance data associated with a user based at least in part on a comparison of the first electrical signal generated by the first electrode and the first electrical signal received by the second electrode; and
transmit a message to a user device associated with the wearable ring device, the message comprising information associated with the first bioimpedance data.

18. The wearable ring device of claim 17, wherein the wearable ring device comprises a plurality of electrodes including the first electrode, the second electrode, and a third electrode, and wherein the first bioimpedance data is associated with a first biological parameter of a plurality of biological parameters, wherein the controller is further configured to:
selectively activate the first electrode and the second electrode from the plurality of electrodes based at least in part on the first biological parameter, wherein generating the first electrical signal, receiving the first electrical signal, or both, are based at least in part on selectively activating the first electrode and the second electrode.

19. The wearable ring device of claim 17, wherein the first bioimpedance data is associated with a first biological parameter, and wherein the first frequency is selected based at least in part on the first biological parameter, wherein the controller is further configured to:
generate a second electrical signal using the first electrode, the second electrode, or both, wherein the second electrical signal is associated with a second frequency selected based at least in part on a second biological parameter;
receive the second electrical signal using a third electrode that is disposed within an outer circumferential surface of the wearable ring device;
determine second bioimpedance data associated with the user associated based at least in part on a comparison of the second electrical signal generated by the first electrode, the second electrode, or both, and the first electrical signal received by the third electrode; and
transmit, to the user device, a second message comprising information associated with the second biological parameter based at least in part on the second bioimpedance data.

20. The wearable ring device of claim 16, wherein the controller is further configured to:
generate, using the first electrode, a plurality of reference electrical signals associated with a plurality of frequencies including the first frequency;
receive the plurality of reference electrical signals using the second electrode;
compare the plurality of reference electrical signals associated with the plurality of frequencies received at the second electrode; and
select the first frequency from the plurality of frequencies based at least in part on the comparison, wherein generating the first electrical signal is based at least in part on selecting the first frequency.
